(19) United States
(12) Patent Application Publication
(10) Pub. No.: US 2004/0110190 A1
(43) Pub. Date: Jun. 10, 2004

(54) Plant cell wall loosening activity of group 2/3 allergens of grass pollen

(76) Inventors: Lian-Chao Li, State College, PA (US); Daniel J. Cosgrove, Pennsylvania Furnace, PA (US)

Correspondence Address: MCKEE, VOORHEES & SEASE, P.L.C., ATTN: PENNSYLVANIA STATE UNIVERSITY, 801 GRAND AVENUE, SUITE 3200, DES MOINES, IA, 50309-2721, US

(21) Appl. No.: 10/628,296

(22) Filed: Jul. 28, 2003

Related U.S. Application Data
(60) Provisional application No. 60/399,688, filed on Jul. 29, 2002.

(51) Int. Cl. C12Q 1/68; C07H 21/04; C12N 9/24; A01H 5/00; C12N 5/04

(52) U.S. Cl. 435/6; 435/69.1; 435/200; 435/320.1; 435/419; 536/23.2; 800/320

(57) ABSTRACT

The present invention provides nucleic acids and polypeptide sequences for a novel class of expansin-related proteins, designated group 2/3 allergen, which comprise the group 2 and group 3 allergens from grass, a purified group 3 allergen Lol p 3, and method of using the nucleic acids sequences and proteins of the invention. Group 2/3 allergens of the invention are significant wall-loosening agents. They are capable of altering cell wall properties, which may effect growth, flexibility, and mechanical strength in tissues in which they are expressed.

### CROSS-REFERENCE TO RELATED APPLICATIONS

This application claims priority under 35 U.S.C. § 120 to provisional application Serial No. 60/399,688 filed Jul. 29, 2002, which is herein incorporated by reference in its entirety.

### GRANT REFERENCE

This work is supported by the Department of Energy pursuant to Grant No. DE-FG02-84ER13179. Accordingly, the U.S. Government may have certain rights in the invention.

### BACKGROUND OF THE INVENTION

Group 2 and group 3 allergens (designated group 2/3 allergens) were first recognized as significant allergenic components of grass pollen in the early 1960's and caused allergenic reaction in about 45˜70% of grass allergic patients. After about a quarter of a century, the complete primary structure of group 2/3 allergen from ryegrass pollen was analyzed by automated Edman degradation. This was soon followed by cDNA cloning from cocksfoot/orchard grass (Dactylis glomerata), timothy grass (Phleum pretense), and perennial ryegrass (Lolium perenne). Up until now, they have been studied exclusively by immunologists concerned with how these proteins elicit hay fever and related allergic responses in humans, but the endogenous activity and role of these proteins have not yet been studied.

For many years wall “loosening enzymes” have been implicated in the control of plant cell enlargement (growth), largely on the basis of rapid biophysical and biochemical changes in the wall during auxin-induced growth (reviewed by Cleland and Rayle, Bot. Mag. Tokyo, 1:125-139, 1978; Taiz, Annu. Rev. Plant Physiol., 35:585-657, 1984). Plant walls contain numerous hydrolytic enzymes, which have been viewed as catalysts capable of weakening the wall to permit turgor-driven expansion (reviewed by Fry, Physiol. Plantarum, 75:532-536, 1989). In support of this hypothesis, Huber and Nevins (Physiol. Plant., 53:533-539, 1981) and Inoue and Nevins (Plant Physiol., 96:426-431, 1991) found that antibodies raised against wall proteins could inhibit both auxin-induced growth and wall autolysis of corn coleoptiles. In addition, isolated walls from many species extend irreversibly when placed under tension in acid conditions in a manner consistent with an enzyme-mediated process (Cosgrove D. J. Planta, 177:121-130, 1989). Despite these results and other evidence in favor of “wall-loosening” enzymes, a crucial prediction of this hypothesis has never been demonstrated, namely, that exogenously added enzymes or enzyme mixtures can induce extension of isolated walls. To the contrary, Ruesink (Planta, 89:95-107, 1969) reported that exogenous wall hydrolytic enzymes could mechanically weaken the wall without stimulating expansion. Similarly, autolysis of walls during fruit ripening does not lead to cell expansion. Thus, a major piece of evidence in favor of wall-loosening enzymes as agents of growth control has been lacking.

Once identified, however, expansins—proteins capable of inducing cell wall extension—would have utility not only in the engineered extension of cell walls in living plants but foreseeably in commercial applications where their chemical reactivity could prove useful. If expansins can disrupt noncovalent associations of cellulose, as they have been shown to do, then they would have particular utility in the paper recycling industry. Paper recycling is a growing concern and will prove more important as the nation's landfill sites become scarcer and more expensive. Paper derives its mechanical strength from hydrogen bonding between paper fibers, which are composed primarily of cellulose. During paper recycling, the hydrogen bonding between paper fibers is disrupted by chemical and mechanical means prior to re-forming new paper products. Proteins which cause cell expansion are thus intrinsically well suited to paper recycling, especially when the proteins are nontoxic and otherwise innocuous, and when the proteins can break down paper products which are resistant to other chemical and enzymatic means of degradation. Use of proteins of this type could thus expand the range of recyclable papers.

Other modes of application of expansins, once they are found, include production of virgin paper. Pulp for virgin paper is made by disrupting the bonding between plant fibers. For the reasons identified above, expansins are useful in the production of paper pulp from plant tissues. Use of expansins can substitute for harsher chemicals now in use and thereby reduce the financial and environmental costs associated with disposing of these harsh chemicals. The use of expansins can also result in higher quality plant fibers because they would be less degraded than fibers currently obtained by harsher treatments.

Thus, a need remains for the identification, characterization, and purification of expansins—proteins which can be characterized as catalysts of the extension of plant cell walls and the weakening of the hydrogen bonds in the pure cellulose paper—and the incorporation of DNA sequences which give rise to such proteins in appropriate expression systems.

### BRIEF SUMMARY OF THE INVENTION

In accordance with the present invention, a family of small proteins (a new class of expansin-like proteins) or a conservatively modified variant thereof and methods related thereto are presented. It should be appreciated that the genes for group 2/3 allergens encode a protein with a signal peptide and a mature protein with significant sequence similarity, up to 42% identity, with domain 2 of expansins, with the greatest similarly to group-1 allergen sub-class of β-expansins. Surprisingly, both the native and recombinant group 2/3 allergens have expansin-like cell wall loosening activity. The proteins of this class can be characterized by the wall-loosening ability in plant tissues and the weakening of the hydrogen bonds in pure cellulose. These small, highly-purified allergens are ˜11 kD nonglycoslyated proteins, and have β-expansin activity. They induce rapid wall extension in extensometer assays of isolated cell walls and also increase the stress relaxation of isolated cell walls over a broad time range. Their activities are saturable, with an acidic pH optimum (pH 5-5.5). Like the group-1 allergen subclass of β-expansins, they have a high specificity of action for grass cell wall over dicot cell wall. Group 2/3 allergens also act synergistically to strongly enhance the wall-loosening activity of group-1 allergen (β-expansin). They also weaken paper, just as expansins do.

These novel proteins reveal a functional activity for group 2/3 allergens of grass pollen and their homologs. These proteins are significant wall-loosening agents in grass pollen and in other tissues where they are expressed.

Because the group 2/3 allergens typically lack cysteines and are active when expressed in bacterial, this form of expansin is a much better candidate for large-scale production and commercialization than α- and β-expansins, which are difficult to express in recombinant protein expression systems. Using standard methods of genetic engineering, which are well known to those of ordinary skill, to express these proteins in bacteria, fungi, and plants, or other systems for protein production, it should be possible to produce large quantities of these proteins for various applications anticipated for expansins, for example, altering the properties of cellulose-based materials such as paper, wood, textiles; wood fiber degradation and biofuel production; and synergistic enhancement of cellulase activity.

Furthermore, because group 2/3 allergens lack cysteines, they are likely to be more stable in commercial applications, i.e., not sensitive to thiol oxidation and inactivation by traces of metals such as mercury, copper, and other oxidative catalysts.

A group 3 allergen, known as Lol p 3 according to the WHO/IUIS Allergen Nomenture Subcomittee (Larsen and Lowenstein, 1999), was purified from ryegrass pollen and examined for the ability to induce wall extension by itself and/or to enhance the wall-loosening activity of β-expansins. The results clearly demonstrate that Lol p 3 possesses expansin-like activity. The proteins of this class include group 2/3 allergens from grass pollen as well as related genes expressed in various tissues, including Tri a 3, a group 2/3-like gene expressed in wheat ovary.

One property of Lol p 3 that is distinct from α-/β-expansin is that dithiothreitol (DTT) has no effect on its wall-loosening activity. This is predictable from its amino acid sequence. It also indicates that there is no contaminant of Lol p 1 in Lol p 3 the preparation.

In addition because the pH-dependent wall loosening activity of Lol p 3 differs from that of the usual class of expansins, this group of proteins will find commercial applications where pH>5 is necessary.

Similar to the activity of α- and β-expansins, it has been found that Lol p 3 also has the following characteristics: First, Lol p 3 could weaken pure cellulose paper, whose strength derives from non-covalent binding between cellulose fibers (FIG. 10A), suggesting that Lol P 3 could also disrupt the hydrogen bonding inside filter paper. Second, in wall-extension reconstitution assays, once wall extension of the heat-inactivated wheat coleoptile was restored by exogenous Lol p 3, the extension could keep going after the bathing solution was changed for one without protein, indicating that added Lol p 3 was tightly bound to the cell wall and continued to exert its action without being released from the cell wall into external solution (FIG. 10B). Third, after heat-inactivated walls pre-treated with Lol p 3 were treated with pronase, a powerful wide-spectrum protease preparation, the reconstituted wall extension activity was completely removed from the walls, whereas control walls that were only incubated with Lol p 3 retained substantial extension activity (FIG. 10C). This suggests that walls treated with Lol p 3 were not permanently altered in their structure so as to become extensible. The results also indicate that the extension activity was due to the action of bound Lol p 3, not to any non-proteinaceious components that might be carried with Lol p 3.

In a first embodiment, there is provided an isolated nucleic acid molecule comprising a polynucleotide selected from the group consisting of (a) a polynucleotide or a its conservatively modified variant thereof having 95% sequence identity to SEQ ID NO:1; (b) a polynucleotide or a conservatively modified variant thereof having the sequence of SEQ ID NO:1; (c) a polynucleotide or a conservatively modified variant thereof encoding a polypeptide having 95% sequence identity to SEQ ID No:2; (d) a polynucleotide or a conservative modified variant thereof that encodes a polypeptide that retains similar biological activity as the unmodified sequence of SEQ ID NO:2; (e) a polynucleotide encoding a polypeptide of SEQ ID NO:2; (f) a polynucleotide that hybridizes under high stringency conditions to the polynucleotide of SEQ ID NO:1; and (g) a polynucleotide complementary to a polynucleotide of (a) through (f).

In another embodiment, there is provided a recombinant expression cassette comprising the described nucleic acid molecule.

In yet another embodiment, there is provided an isolated nucleic acid comprising a polynucleotide sequence encoding a polypeptide selected from the group consisting of: SEQ ID NO:2, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18.

In yet another embodiment, there is provided an isolated polypeptide comprising a polypeptide selected from the group consisting of: (a) a polypeptide or a conservatively modified variant thereof having an amino acid sequence 95% identical to the amino acid sequence of SEQ ID NO:2; (b) a polypeptide or a conservatively modified variant thereof having the amino acid sequence of SEQ ID NO:2; (c) a polypeptide or a conservatively modified variant thereof that retains similar biological activity as the unmodified sequence of SEQ ID NO:2; and (d) a polypeptide which is encoded by the polynucleotide of SEQ ID NO:1.

In yet another embodiment, there is provided an antibody which selectively binds to the described polypeptides.

In yet another aspect, an embodiment of the invention relates to a method of altering physical properties of the plant cell wall or any cell wall products derived from plant material, for example, paper or textile.

In a further aspect, an embodiment relates to a method of identifying, isolating, and purifying an expansin protein or a polynucleotide encoding such protein.

These and other objects, features, and advantages of the present invention will become apparent after review of the following detailed description of the disclosed embodiments and the appended claims.

### BRIEF DESCRIPTION OF THE DRAWINGS

FIG. 1 depicts the purification of β-expansins and group 2/3 allergens from ryegrass pollen. (A) shows fractionation of ryegrass pollen extract by SP-Sepharose cation exchange chromatography. Crude ryegrass pollen extract was loaded onto a SP-Sepharose Fast Flow column (10×200 mm) and equilibrated in 20 mM sodium acetate, pH 4.5. The column was washed with the same buffer until baseline was reached, and then a 0˜500 mM NaCl linear gradient in 2 hour hold at final gradient (500 mM NaCl) for 1 hour was applied to the column to wash all other bound proteins at the flow rate of 0.75 mL/min. (B) shows the active fractions from the previous step that were further purified on a silica-based CM-HPLC column. The fractions from SP-Sepharose column chromatography were desalted and concentrated by ultrafiltration. Active fractions were pooled and filtrated through the low binding Durapore membrane (0.45 μm), and then loaded onto a silica-based CM-HPLC column. Proteins were eluted at 1 mL/min with a 0˜650 mM NaCl, 20 mM sodium acetate, pH 4.5, linear gradient in 50 minutes. (C) and (D) show the final purification of β-expansins and group 2/3 allergens by high performance gel filtration chromatography. Fractions from the CM-HPLC step were desalted and concentrated as described above, and those containing expansin-like activity were further subjected to high-performance gel filtration chromatography. Proteins isocratically eluted from the two coupled columns with 200 mM NaCl, 20 mM sodium acetate, pH 4.5, were assayed for expansin-like activity. Proteins eluted from the columns were monitored at 280 nm.

FIG. 2 depicts an SDS-PAGE and immunoblot of β-expansins and group 2/3 allergens. In (A) and (C), approximately 5 μg of β-expansins and group 2/3 allergen were loaded into 12% and 15% SDS-PAGE gel respectively. Gels were stained with Coomassie Brilliant Blue R-250 after electrophoresis. (B) and (D) depicts nitrocellulose membranes containing the electrophoretically transferred β-expansins and group 2/3 allergens from SDS-PAGE gels were blocked with 10% horse serum and incubated with mouse monoclonal antibody raised against Lol p 1 and rabbit polyclonal antibody against Lol p 2, respectively, then incubated with the secondary antibody (goat anti-mouse IgG-alkaline phosphatase conjugate or goat anti-rabbit IgG (heavy and light chains)-conjugated alkaline phosphatase). Immuno-specific bands were developed with NBT and BCIP as phosphatase substrate. Lane M1: Navagen perfect protein marker; Lane L1: Lol p 1; Lane M2: GIBCO BRL prestained protein ladder; Lane M3: GIBCO BRL regular low molecular weight protein standards; Lane L3: Lol p 3; Lane M4: GIBCO BRL prestained low molecular weight protein standards.

FIG. 3 depicts the cDNA (SEQ ID NO:1) and deduced amino acid sequence (SEQ ID NO:2) of ryegrass Lol p 3.

FIG. 4 shows reconstitution of wall extension activity with group 2/3 allergens. Heat-inactivated cucumber hypocotyl and wheat coleoptile walls from the etiolated seedlings or maize silk walls from the field plants were mounted on a custom-made extensometer. After initial extension in 50 mM sodium acetate, pH 4.5, for 30 minutes, the bathing solution was replaced with one containing HPGFC purified natural (A) Lol p 3 (50 μg/mL) and (B) Phl p 2/3 (50 μg/mL), and (C) CM-Sepharose chromatographic purified recombinant Lol p 3 (250 μg/mL) which was expressed in E. coli cells. In (B) and (C), only heat-inactivated wheat coleoptiles were used for assaying β-expansin-like activity. In (C), inactivated wall segments were treated with either partially purified recombinant Lol p 3 or the same amount of equivalent protein in untransformed E. coli cells. Similar results were obtained in five independent experiments.

FIG. 5 shows substrate preferences of group 2/3 allergen. Heat-inactivated Type II (A) and Type I (B) walls from etiolated plant seedlings were mounted on a custom-made extensometer. After 30 minutes of extension in 50 mM sodium acetate, pH 4.5, the bathing solution was replaced with one containing HPGFC purified Lol p 3 (50 μg/mL). Walls resistant to Lol p 3 were verified to be extensible in the above buffer when the heat treatment step was omitted. The extension activity was calculated by subtracting the baseline rate before Lol p 3 addition from the rate after the addition of the protein, and expressed as a % increase in length per hour above the baseline. The negative values arise in some Type I walls in (B) because of mechanical weakening of the walls, which leads to higher initial extension rates. Data presented are the means (+/−SE) extension activity of at least four experiments for each wall.

FIG. 6 shows the wall stress relaxation spectrum by group 2/3 allergens. Heat-inactivated walls were pretreated for 10 minutes in either 50 mM sodium acetate, pH 4.5 or one containing 0.05 mg/mL group 2/3 allergen, and were held between two special clamps (5 mm between the jaws) in a custom-made tensile tester. Walls were relaxed at a rate of 170 mm/min until a stress of 20 g was attained and then were held at a constant strain. The relaxation spectrum was calculated as the derivative of the stress with respect to log (time). Each relaxation curve is the average of 10 independent relaxation measurements.

FIG. 7 shows the synergistic effect between Lol p 1 and Lol p 3 on wall extension activity. Heat-inactivated wheat coleoptile walls were mounted on a custom-made extensometer. After 30 minutes of extension in 50 mM sodium acetate, pH 4.5, the bathing solution was replaced with one containing minimal amounts of HPGFC purified β-expansin, group 2/3 allergen, or a combination of both. Data are the means+/−SE (n=5).

FIG. 8 shows the dependence of the wall-loosening activity of Lol p 3 on pH. Heat-inactivated walls from wheat coleoptile were initially bathed in 50 mM 3,3-dimethylglutaric acid buffer at different pHs. After 30 minutes the bathing solutions were replaced with 0.2 mL of the corresponding buffer containing the same amount of Lol p 3 (10 μg). Data are the means+/−SE (n=5).

FIG. 9 shows the effect of concentration of Lol p 3 on its wall-loosening activity. Heat-inactivated walls from wheat coleoptile were first incubated in 50 mM 3,3-dimethylglutaric acid, pH 5.5. After 30 minutes of extension, the external solution was replaced with 0.2 mL of the same buffer containing a different amount of Lol p 3. Data are the means+/−SE (n=5).

FIG. 10 shows the characteristics of wall-loosening activity of Lol p 3. (A) shows the disruption of hydrogen bonding between cellulose fibers. Strips of Whatman No. 3 filter paper were clamped in an extensometer in 50 mM sodium acetate, pH 4.5. After 30 minutes the buffer was replaced with 0.2 mL of the same buffer containing 10 μg of Lol p 3. The control contained no protein addition. (B) shows the independence of presence of the external Lol p 3 in reconstituted extension of wheat coleoptile wall. After 30 minutes of extension of the heat-inactivated wheat coleoptile wall in 50 mM sodium acetate, pH 4.5, the incubation solution was replaced with one containing 50 μg/mL Lol p 3, and the wall extended for a further 20 minutes. The wall and the cuvette of the extensometer were then washed thoroughly with the buffer and the external solution was changed back to the initial buffer without Lol p 3. The negative control contained no Lol p 3 addition, while the positive control continued extending in 50 μg/mL Lol p 3 solution. (C) depicts destruction of group 2/3 allergen activity by Pronase. Heat-inactivated wheat coleoptiles were first incubated for 1 hour with 100 μg/mL of Lol p 3 in 50 mM sodium acetate, pH 4.5, after 5 washes with 50 mM Mes, 1 mM EDTA, 5 mM DTT, pH 6.0, and they were further treated for another one hour at room temperature with 2 mg/mL of Pronase in the washing buffer. The above treated walls were then mounted on an extensometer. After 30 minutes of incubation in 50 mM Hepes, pH 6.8, the bathing buffer was switched to 50 mM sodium acetate, pH 4.5. In the positive control, Pronase treatment was replaced by incubation of the wall with washing buffer without Pronase. All of the experiments were performed at least five times with similar results.

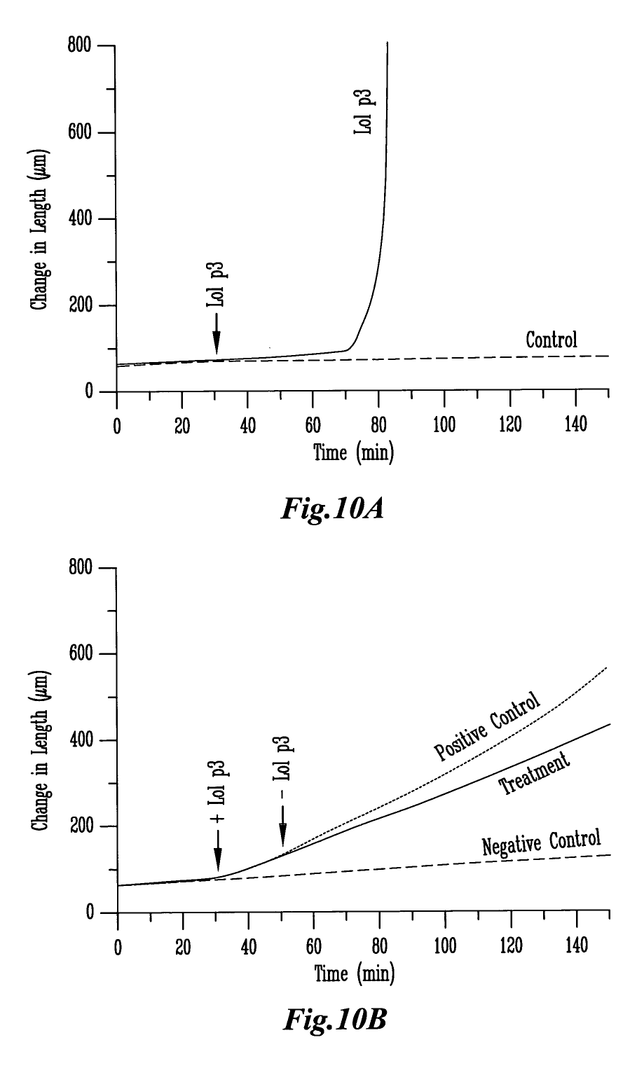

FIG. 11 depicts a conservatively modified variant of SEQ ID NO:1.

FIG. 12 depicts a conservatively modified variant of SEQ ID NO:2.

FIG. 13 shows related to amino acids.

### DETAILED DESCRIPTION OF THE PREFERRED EMBODIMENT

Although group 2/3 allergens from grass pollen have been studied for many years by immunologists concerned with how they elicit hay fever and related allergic responses in humans, the native activity and biological roles of these proteins have not been examined. These small (˜11 kD) proteins have sequence similarity to the C-terminus of expansins. Expansins induce tension and stress relaxation of plant cell walls in a unique manner and function in a variety of plant developmental processes where cell wall loosening is important. Group 2/3 grass pollen allergens are distinguished by pI and immuno-cross reactivity, but sequence information indicates that they belong to the same protein family, referred to herein as group 2/3 allergens.

Two families, α- and β- of expansins are currently recognized, and group 2/3 allergens are closest in sequence to the subset β-expansins known to immunologists as the grass pollen group 1 allergens. α- and β- of expansins, each contain an N-terminal domain, homologous with the catalytic domain of glycosyl hydrolase family 45 (GH45) enzymes, and a C-terminal domain, hypothesized to be a polysaccharide-binding domain.

The characteristic action of expansin on cell wall extensibility and stress relaxation is principally due to the action of domain 2. However, except for the single case of the grass group-2/3 pollen allergens, the GH45-like domain 1 of expansin has apparently been preserved throughout plant evolution (˜500 million years). This in turn implies that the GH45-like domain has an important role in expansin function.

In accordance with the present invention, there are provided polynucleotides encoding group 2/3 allergens, a novel class of proteins, polypeptides encoded by the described polynucleotides, vectors, and host cells containing such polypeptides. It should be appreciated that previously, α- and β-expansins have not been able to be folded properly in an expression system. Surprisingly, however, Applicants have shown that recombinant group 2/3 allergens have expansin-like cell wall loosening activity. The proteins can be characterized as catalysts of the extension of plant cell walls and the weakening of the hydrogen bonds in pure cellulose.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present invention, the following terms will be employed, and are intended to be defined as indicated below and herein.

By “altering physical characteristics of a plant cell wall” includes loosening or expanding cell walls, altering cell wall mechanical strength, altering the bonding relationship between the components of the cell wall and/or altering the growth of the plant cell wall.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Proc. Natl. Acad. Sci. (USA), 82: 2306-2309 (1985)), or the ciliate Macronucleus, may be used when the nucleic acid is expressed using these organisms.

By “host cell” or “recombinantly engineered cell” is meant a cell, which contains a vector and supports the replication and/or expression of the expression vector. Host cells may be prokaryotic cells such as E. coli, or eukaryotic cells such as yeast, Pichia, insect, plant, amphibian, or mammalian cells.

The present invention provides expression vectors and host cells transformed to express the nucleic acid sequences of the invention. Nucleic acid coding for these group 2/3 allergens, or at least one fragment thereof may be expressed in bacterial cells such as E. coli, fungi cells, plants or other systems for protein production. Suitable expression vectors, promoters, enhancers, and other expression control elements may be found in Sambrook et al. Molecular Cloning: A Laboratory Manual, second edition, Cold Spring Harbor Laboratory Press, Cold Spring Harbor, N.Y., 1989, which is incorporated herein by reference in its entirety.

Expression vectors are typically self-replicating DNA or RNA constructs containing the desired receptor gene or its fragments, usually operably linked to suitable genetic control elements that are recognized in a suitable host cell. These control elements are capable of effecting expression within a suitable host. The specific type of control elements necessary to effect expression will depend upon the eventual host cell used. Generally, the genetic control elements can include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. Expression vectors also usually contain an origin of replication that allows the vector to replicate independently of the host cell.

The vectors of this invention include those which contain DNA which encodes a protein, as described, or a fragment thereof encoding a biologically active equivalent polypeptide. The DNA can be under the control of a bacterial promoter and can encode a selection marker. Usually, expression vectors are designed for stable replication in their host cells or for amplification to greatly increase the total number of copies of the desirable gene per cell. It is not always necessary to require that an expression vector replicate in a host cell, e.g., it is possible to effect transient expression of the protein or its fragments in various hosts using vectors that do not contain a replication origin that is recognized by the host cell. It is also possible to use vectors that cause integration of the protein encoding portion or its fragments into the host DNA by recombination.

Expression vectors are specialized vectors which contain genetic control elements that effect expression of operably linked genes. Plasmids are the most commonly used form of vector but all other forms of vectors which serve an equivalent function and which are, or become, known in the art are suitable for use herein. See, e.g., Pouwels, et al. (1985 and Supplements) Cloning Vectors: A Laboratory Manual, Elsevier, N.Y., and Rodriguez, et al. (eds. 1988) Vectors: A Survey of Molecular Cloning Vectors and Their Uses, Buttersworth, Boston, which are incorporated herein by reference in their entirety.

The expression vectors listed herein are provided by way of example only of the well-known vectors available to those of ordinary skill in the art that would be useful to express the nucleic acid molecules. The person of ordinary skill in the art would be aware of other vectors suitable for maintenance propagation or expression of the nucleic acid molecules described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

Prokaryotic host-vector systems include a wide variety of vectors for many different species. As used herein, E. coli and its vectors will be used generically to include equivalent vectors used in other prokaryotes. A representative vector for amplifying DNA is pBR322 or many of its derivatives. Vectors that can be used to express the polynucleotide or, its fragments include, but are not limited to, such vectors, as those containing the tac, ara, trp promoter, lac promoter, lacUV5 or T7 promoter. See Brosius, et al. (1988) “Expression Vectors Employing Lambda-, trp-, lac-, and Ipp-derived Promoters”, in Vectors: A Survey of Molecular Cloning Vectors and Their Uses, (eds. Rodriguez and Denhardt), Buttersworth, Boston, Chapter 10, pp. 205-236, which is incorporated herein by reference. Moreover, one skilled in the art knows that such microorganisms are available from depository authorities, e.g., the American Type Culture Collection (ATCC).

The use of promoter and cell type combinations for protein expression is generally known to those of skill in the art of molecular biology, for example, see Sambrook et al (1989). The promoters employed may be constitutive, or inducible, and can be used under the appropriate conditions to direct high level expression of the introduced DNA segment, such as is advantageous in the large-scale production of recombinant proteins or peptides.

Host cells can be transformed to express the nucleic acid sequences of the invention using conventional techniques such as calcium phosphate or calcium chloride co-precipitation, DEAE-dextran-mediated transfection, or electroporation. Suitable methods for transforming the host cells may be found in Sambrook et al. supra, which is incorporated by reference in its entirety and other laboratory textbooks.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “residue” or “amino acid residue” or “amino acid” is used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

The term “expression cassette” refers to a polynucleotide sequence that comprises the coding sequence of interest and regulatory elements which affect expression of the protein of interest. Typically, expression cassettes include a promoter, the coding sequence of interest, a termination sequence, and a polyadenylation sequence. Optionally, expression cassettes can include enhancer elements and other regulatory elements.

The term “isolated nucleic acid” refers to a nucleic acid which is essentially free of other cellular components with which it is associated in the natural state. It is preferably in a homogeneous state although it can be in either a dry or aqueous solution. Purity and homogeneity are typically determined using analytical chemistry techniques such as gel electrophoresis or high performance liquid chromatography. The term “purified” denotes that a nucleic acid gives rise to essentially one band in an electrophoretic gel. Particularly, it means that the nucleic acid is at least 85% pure, more preferably at least 95% pure, and most preferably at least 99% pure.

The term “modifying or modification of cell walls” refers to changing the components, ratio of the components or structure of the components present in the cell wall, e.g., interference with the covalent interactions between cellulose microfibrils and matrix polysaccharides (McQueen-Mason, S. J. and Cosgrove, D. J. Plant Physiol. 107:87 (1995).

The term “operably linked” refers to functional linkage between a promoter and a second sequence, wherein the promoter sequence initiates transcription of RNA corresponding to the second sequence.

The sequence of the molecule can be defined herein in terms of homology to the exemplified sequence as well as in terms of the ability to hybridize with, or be amplified by, certain exemplified probes and primers. The polypeptides provided herein can also be identified based on their immunoreactivity with certain antibodies.

The polypeptides and polynucleotides of the subject invention can be identified and obtained by using oligonucleotide probes, for example, these probes are detectable nucleotide sequences. The probes (and the polynucleotides of the subject invention) may be DNA, RNA, or PNA (peptide nucleic acid). These sequences may be detectable by virtue of an appropriate label or may be made inherently fluorescent as described in International Application No. WO93/16094. As is well known in the art, if the probe molecule and nucleic acid sample hybridize by forming a strong bond between the two molecules, it can be reasonably assumed that the probe and sample have substantial homology. Preferably, hybridization is conducted under stringent conditions by techniques well-known in the art, as described, for example, in Keller, G. H., M. M. Manak (1987) DNA Probes, Stockton Press, New York, N.Y., pp. 169-170.

The term “polynucleotide,” “polynucleotide sequence” or “nucleic acid sequence” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, the nucleic acid sequence of this invention also implicitly encompasses conservatively modified variants thereof (e.g., degenerate codon substitutions) and complementary sequences and as well as the sequence explicitly indicated.

As used herein, the term “equivalent polypeptides” refers to polypeptides having the same or essentially the same biological activity as the claimed polypeptide.

As used herein, the terms “variants” or “variations” of genes refer to nucleotide sequences which encode the same polypeptides or which encode equivalent polypeptides.

Because of the redundancy of the genetic code, a variety of different DNA sequences can encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine, one exception is Micrococcus rubens, for which GTG is the methionine codon (Ishizuka, et al., J. Gen'l Microbiol, 139:425-432 (1993)) can be modified to yield a functionally identical molecule. Accordingly, each silent variation of a nucleic acid, which encodes a polypeptide of the present invention, is implicit in each described polypeptide sequence and incorporated herein by reference.

It is well within the skill of a person trained in the art to create these alternative DNA sequences encoding the same, or essentially the same polypeptide. These variant DNA sequences are within the scope of the subject invention. As used herein, reference to “essentially the same” sequence refers to sequences which have amino acid substitutions, deletions, additions, or insertions which do not materially affect activity. Fragments retaining activity are also included in this definition.

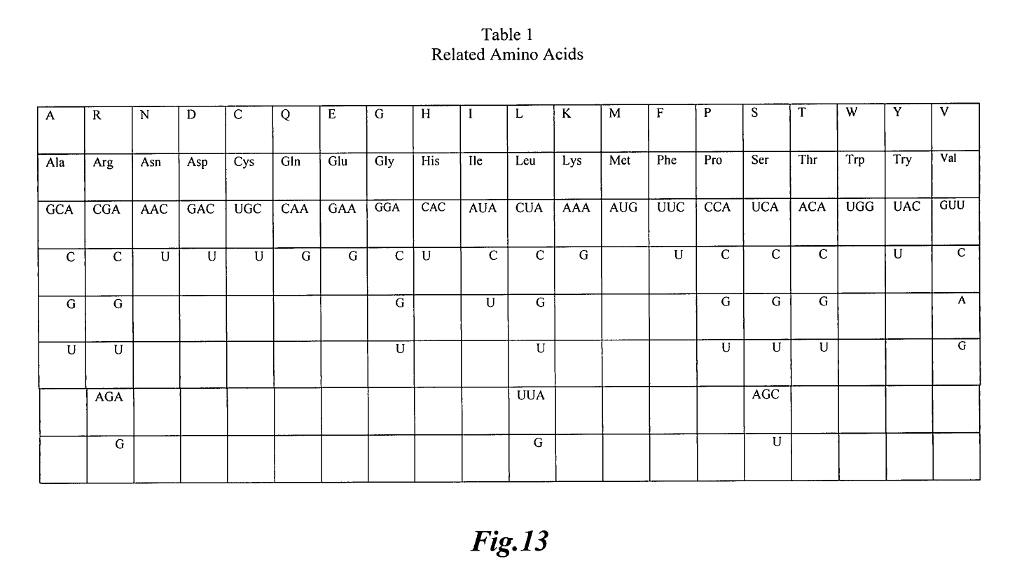

For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Other conservatively modified variants may be derived using FIG. 13, which shows related amino acids.

Every nucleic acid sequence herein that encodes a polypeptide also describes every possible silent variation of the nucleic acid which encodes a polypeptide of the present invention, is implicit in each described polypeptide sequence and incorporated herein by reference.

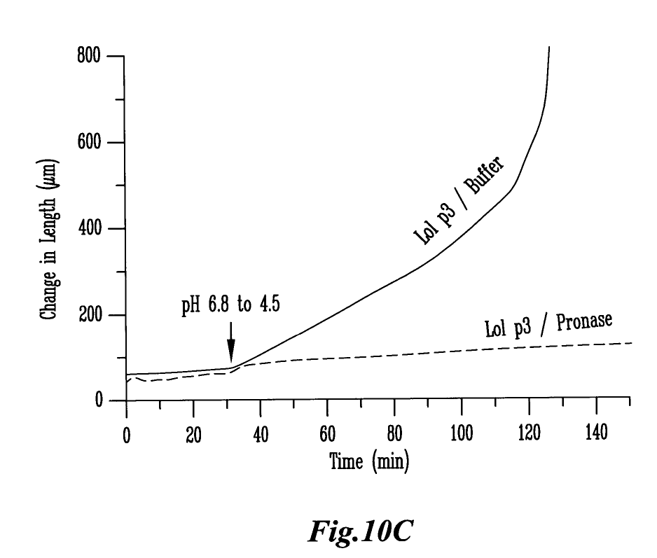

A “silent variation” of SEQ ID NO:1 may be achieved by generating a variant sequence (SEQ ID NO:3) as set for forth in FIG. 11 wherein the bolded letters denote a substituted nucleotide (proline; CCA→proline; CCC) which has not altered the encoded polypeptide.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90%, preferably 60-90% of the native protein for it's native substrate.

The amino acid homology will be highest in critical regions of the polypeptides which account for biological activity or are involved in the determination of three-dimensional configuration which ultimately is responsible for the biological activity. In this regard, certain amino acid substitutions are acceptable and can be expected if these substitutions are in regions which are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. Table 1 provides a listing of examples of amino acids belonging to each class.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glw |
| Basic | Lys, Arg, His |

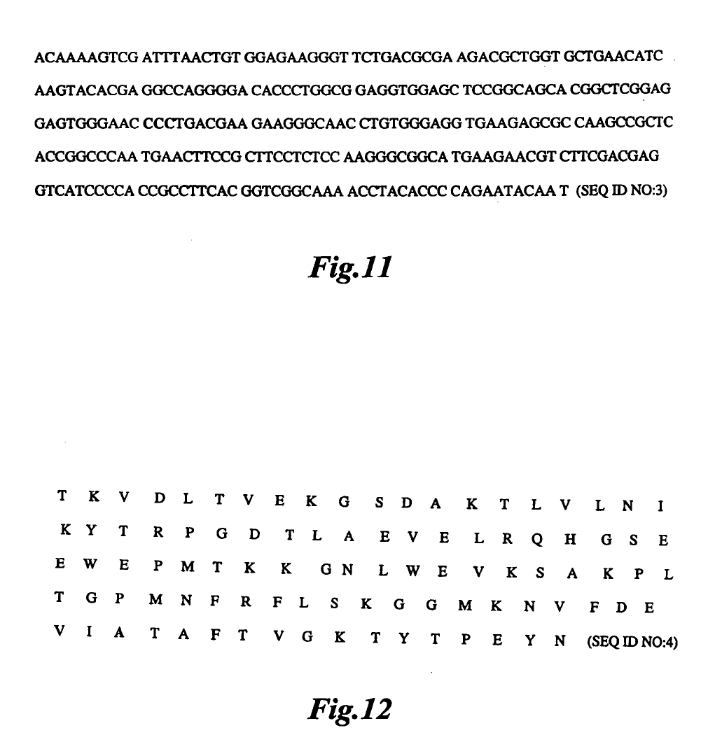

In some instances, non-conservative substitutions can also be made. The critical factor is that these substitutions must not significantly detract from the biological activity of the polypeptides. Such an example is set forth in FIG. 12 wherein the bolded amino acid (A) denotes a conservative substitution whereby an amino acid of one class is replaced with another amino acid of the same type (Proline, CCA→Alanine, GCA).

Synthetic genes which are functionally equivalent to the polynucleotides of the subject invention can also be used to transform hosts. Methods for the production of synthetic genes can be found in, for example, U.S. Pat. No. 5,380,831. See also, Creighton (1984) Proteins W. H. Freeman and Company.

Equivalent polypeptides will have amino acid homology with exemplified polypeptides. The amino acid identity will typically be greater than 60%, preferably be greater than 70%, more preferably greater than 80%, more preferably greater than 90%, and can be greater than 95%.

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (Best Fit) of Smith and Waterman, Adv. Appl. Math may conduct optimal alignment of sequences for comparison. 2: 482 (1981); by the homology alignment algorithm (GAP) of Needleman and Wunsch, J. Mol. Biol. 48: 443 (1970); by the search for similarity method (Tfasta and Fasta) of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994). The preferred program to use for optimal global alignment of multiple sequences is PileUp (Feng and Doolittle, Journal of Molecular Evolution, 25:351-360 (1987) which is similar to the method described by Higgins and Sharp, CABIOS, 5:151-153 (1989) and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

GAP uses the algorithm of Needleman and Wunsch (J. Mol. Biol. 48: 443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50, or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff (1989) Proc. Natl. Acad. Sci. USA 89:10915).

Unless otherwise stated, sequence identity/similarity values provided herein refer to the value obtained using the BLAST 2.0 suite of programs using default parameters. Altschul et al., Nucleic Acids Res. 25:3389-3402 (1997).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 40-100%, preferably at least 55%, preferably at least 60%, more preferably at least 70%, 80%, 90%, and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, J. Mol. Biol. 48: 443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The term “transgenic plant” refers to a plant into which exogenous polynucleotides have been introduced by any means other than sexual cross or selfing. Examples of means by which this can be accomplished are described below, and include Agrobacterium-mediated transformation, biolistic methods, electroporation, in planta techniques, and the like. Such a plant containing the exogenous polynucleotides is referred to here as an R.sub.1 generation transgenic plant. Transgenic plants may also arise from sexual cross or by selfing of transgenic plants into which exogenous polynucleotides have been introduced.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-95% sequence identity, and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. One of ordinary skill is apprised in knowing that the time of the hybridization is dependent on the concentration of the probe. Exemplary low stringency conditions include hybridization with a buffer solution of 30 to 35% formamide, 1 M NaCl, 1% SDS (sodium dodecyl sulphate) at 37° C., and a wash in 1× to 2× SSC (20× SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1× SSC at 55 to 60° C. Exemplary high stringency conditions include hybridization in 50% formamide, 1 M NaCl, 1% SDS at 37° C., and a wash in 0.1× SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6(log M)+0.41(% GC)−0.61(% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995). Unless otherwise stated, in the present application high stringency is defined as hybridization in 4× SSC, 5× Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1× SSC, 0.1% SDS at 65° C., two to three times for at least 15 minutes.

Purification of β- and Group 2/3 Allergens

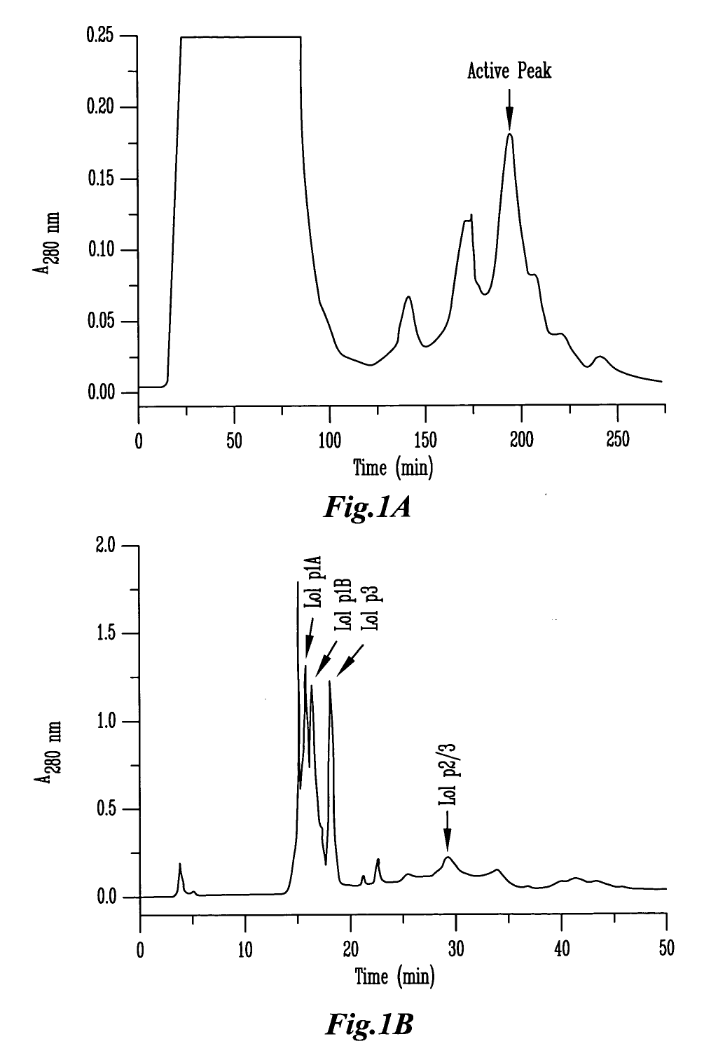

Purification of β-expansins and group 2/3 allergens from ryegrass pollen involved three successive chromatographic steps as depicted in FIG. 1. During the purification steps, fractions from each step were examined for expansin-like proteins by wall extension assay in combination with SDS-PAGE. The starting material was crude extract obtained from commercial ryegrass pollen with 50 mM sodium acetate, pH 4.5. On the conventional SP-Sepharose cation exchange chromatographic column (FIG. 1A), the proteins with expansin-like activity were well separated from unbound impurities, yielding a sharp peak which predominantly contained expansin activity-like proteins. The fractions under this peak were pooled, desalted/concentrated through a 5 kD cutoff filtration membrane, and then chromatographed on a CM-silica based HPLC column. β-expansins were eluted in two peaks (designated to Lol p 1A and Lol p 1B), followed by a low molecular weight (LMW) expansin-like protein peak (Lol p 3) when a linear salt gradient was applied (FIG. 1B). Fractions containing β-expansins (Lol p 1A and Lol p 1B) and group 2/3 allergen (Lol p 3) were concentrated and desalted as described above and then were separately loaded into two coupled high-performance gel filtration column which have different exclusion limits. In both cases, a major symmetrical peak was obtained, which was identified as pure Lol p 1 or Lol p 3 (FIG. 1C and FIG. 1D) as described below.

Identification of β-Expansins and Group 2/3 Allergens

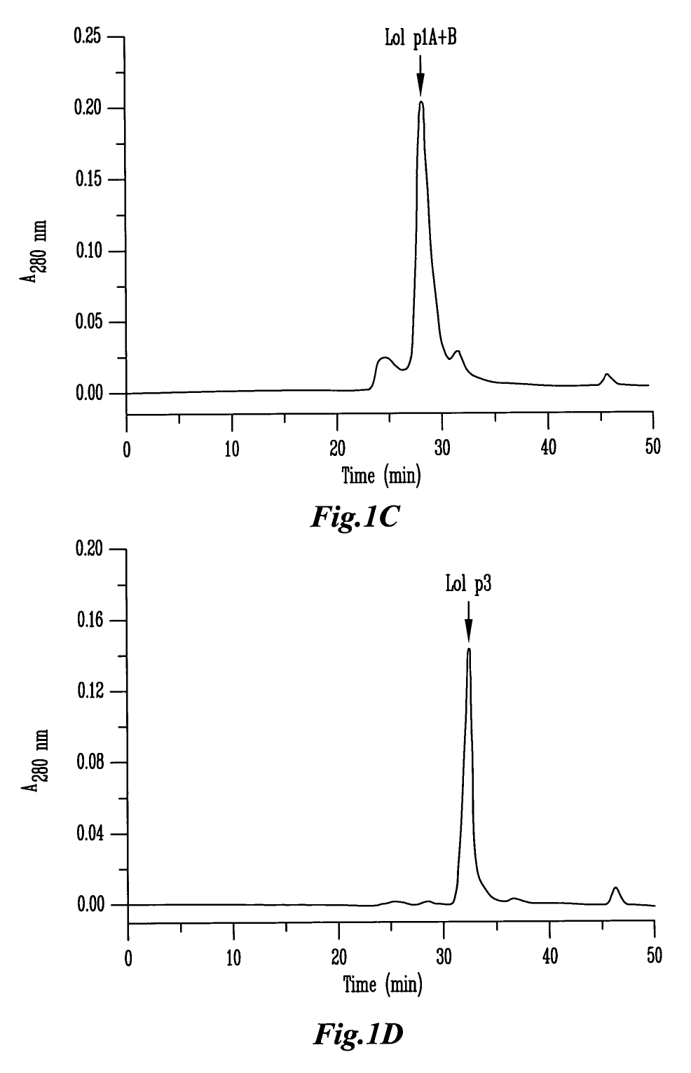

SDS-PAGE analysis shows that both purified β-expansin Lol p 1 and the group 2/3 allergen Lol p 3 gave a single band at appropriate gel positions which correspond to their reported molecular weight (FIGS. 2A/C). Western blots revealed that Lol p 1 and Lol p 3 were recognized by an authentic monoclonal antibody which was raised against Lol p 1 in mouse (FIG. 2B) and rabbit polyclonal antibody against Lol p 2 (FIG. 2D), respectively. For further identification, the molecular mass of the LMW expansin-like protein was measured by both matrix-assisted laser desorption/ionization time-of-flight (MALDI-TOF) and electrospray ionization time-of-flight (ESI-TOF) mass spectrometry, giving a molecular weight of 10,884.2 (data not shown) and 10,908.1 dalton (data not shown), respectively, which are in excellent agreement with the calculation of 10,879.2 dalton from the complete amino acid sequence of Lol p 2 and that of 10,908.8 dalton from Lol p 3 protein sequence. These results indicate that the LMW expansin-like protein from rye grass pollen is a group-2 allergen (Lol p 2) or a group-3 allergen (Lol p 3).

Identification of Lol p 3 Via its N-Terminal Amino Acid Sequence

To further identity whether the LMW expansin-like protein is Lol p 2 or Lol p 3 and to obtain necessary information for its gene cloning, the highly purified protein was analyzed for its N-terminal amino acid sequence. The resultant data revealed that the first 20 amino acid residues at the N-terminus were the following, using the one-letter amino acid code: TKVDLTVEKGSDAKTLVLNI (SEQ ID NO:5), which matches exactly with the amino acid sequence of Lol p 3 reported in the literature (Ansari et al., 1989) and in the protein sequence databank (Accession No. P14948). Therefore, the purified LMW expansin-like protein described herein was definitively identified as group-3 allergen Lol p 3 rather than the group-2 allergen Lol p 2 by its N-terminal sequence and its molecular mass.

cDNA Cloning of Lol p 3

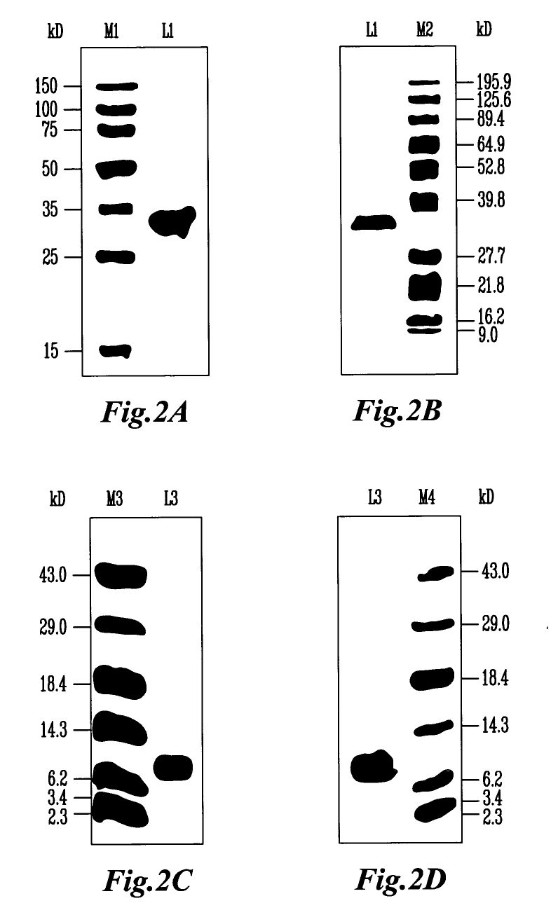

Sense and anti-sense primers, corresponding respectively to the N- and C-terminal amino acid sequence of Lol p 3, were synthesized and used in a temperature gradient polymerase chain reaction (PCR) with ryegrass genomic DNA as the template. An approximately 300 bp DNA fragment was obtained, cloned, and sequenced. The deduced amino acid sequence of the PCR product was identical with the primary structure of Lol p 3 which was directly determined by the automated Edman degradation of the Lol p 3 protein. FIG. 3 shows the nucleotide sequence of the cDNA (SEQ ID NO:1) and the deduced amino acid sequence (SEQ ID NO:2).

Effects of Group 2/3 Allergens on Wall Rheological Properties

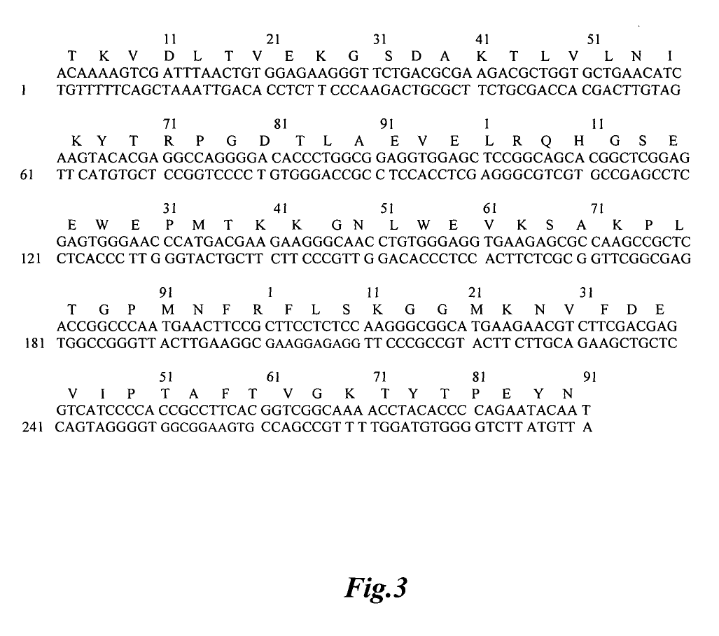

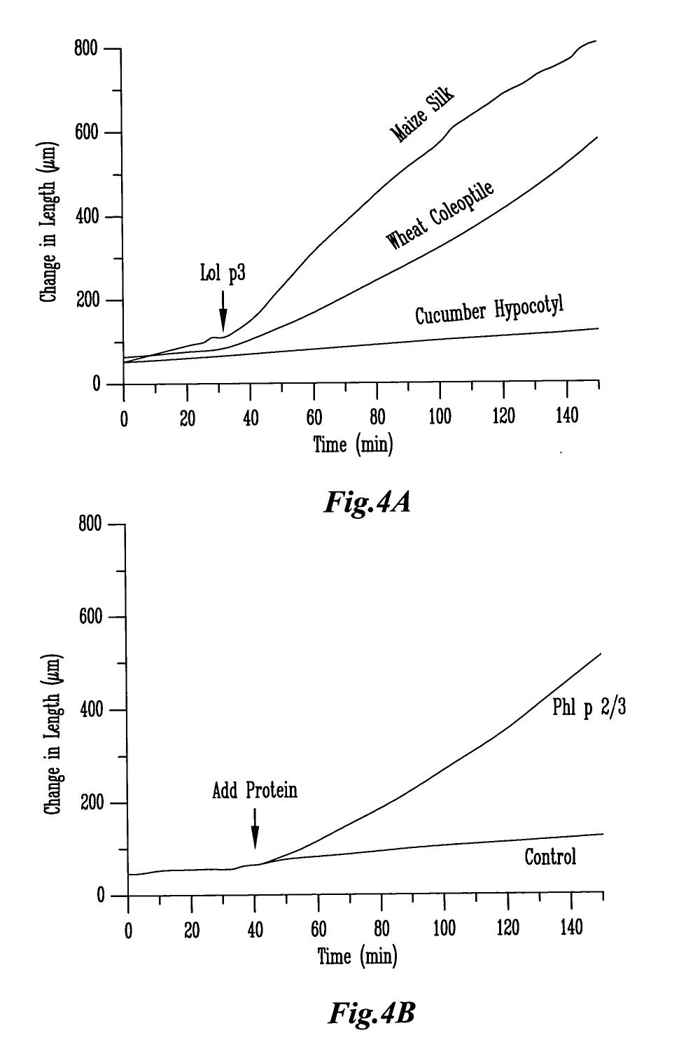

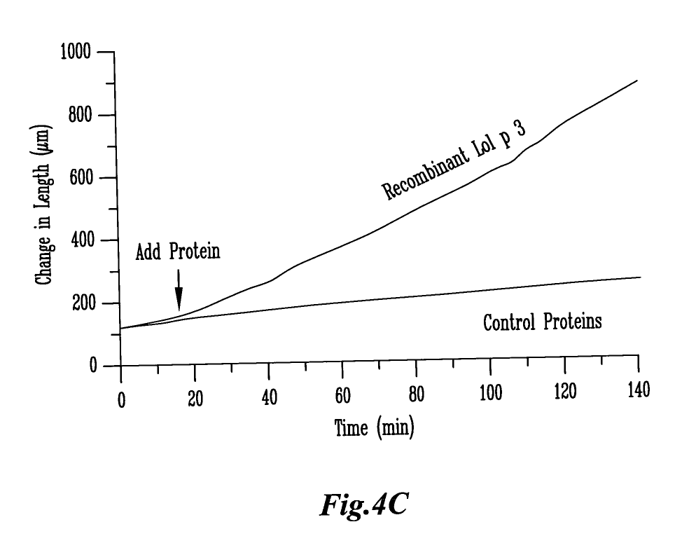

Wall extension reconstitution experiments showed that highly purified Lol p 3, by itself, could induce extension of plant cell walls from maize silks, the natural substrate of β-expansin Zea m 1, and from wheat coleoptiles, whereas it is completely inactive on cucumber hypocotyls cell walls (FIG. 4A). This phenomenon was confirmed and extended by use of other native proteins in this family, such as Lol p 2/3 and Phl p 2/3 (FIG. 4B), and especially by recombinant Lol p3 which was expressed in a bacterial system (FIG. 4C). Likewise, other grass coleoptile walls from maize (Zea mays L.) barley (Hordeum sativum L.), rice (Oryza sativa L.), and oat (Avena sativa L.) are also substantially responsive in these reconstitution assays, whereas hypocotyls walls from dicot plants, such as soybean (Glycine max L.), tomato (Lycopersicon esculentum Mill.), rape (Brassica napus L.), pea (Pisum sativum L.) and pepper (Capsicum annuum L.) are inert to the action of these proteins (FIG. 5). In the stress-relaxation experiments, when heat-inactivated wheat coleoptile or maize silk walls were briefly incubated with Lol p 3, the wall relaxation rates were obviously enhanced, as compared with the boiled walls used as controls. However, the patterns of their stress-relaxation spectra looked very similar (FIG. 6). No effect was observed on cucumber wall. These results were similar to the results obtained using group-1 allergen Zea m 1. Taken together, the conclusion was that the group 2/3 allergens possess wall-loosening activity characteristic of expansins.

Wall extension activity of Lol p 3 was saturable and was most active at pH 5.5, which is close to the wall pH of many plant tissues. The sharp reduction in activity at pH<4.5 is very different from that found for β-expansins, which have higher activity at pH 3-4 than at pH 5-6. This property is germane to the “acid growth” process in plants, wherein plant cell enlargement and wall loosening are stimulated by low cell wall pH. Because of its pH dependence, Lol p 3 is not a good candidate as an acid-growth agent. Instead, its pH dependence is centered on the normal pH of the cell wall.

Synergistic Action Between β-expansins and Group 2/3 Allergens

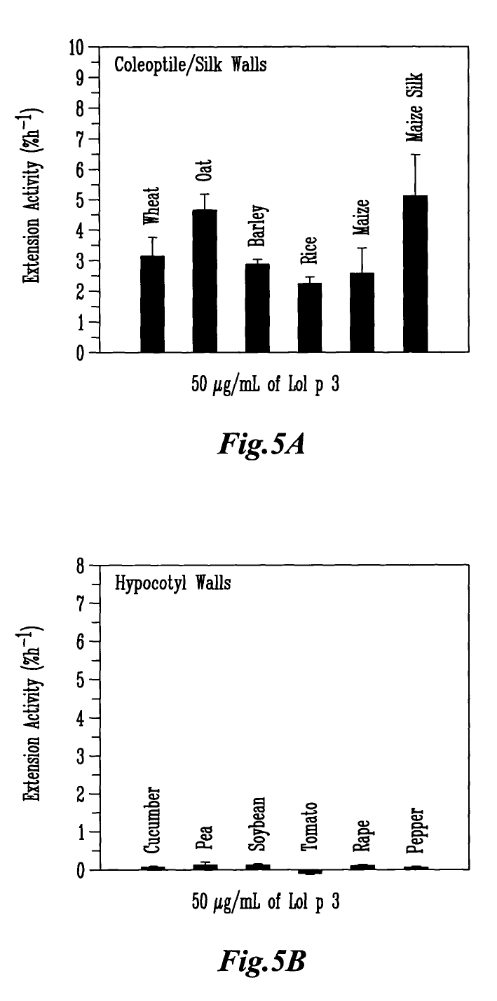

The actions of β-expansin Lol p 1 and group 2/3 allergen Lol p 3, applied together, were found to synergistically enhance each other in assays of grass wall extension and stress-relaxation. In the wall extension reconstitution assays, Lol p 1 and Lol p 3 had synergistic effects on wheat coleoptile wall-loosening when the minimal amounts of each protein at a 1:1 molar ratio was used (FIG. 7). Similarly, when wheat coleoptiles were reconstituted with a combination of Lol p 1 and Lol p 3 at the same ratio, their effects on its wall stress-relaxation mode were synergistic rather than additive. This further confirmed that they caused significant synergistic effect on wall extension in vitro as shown in FIG. 7. However, the striking synergism observed on grass walls was not seen with dicot cell walls.

Characterization of Lol p 3 Wall-Loosening Activity

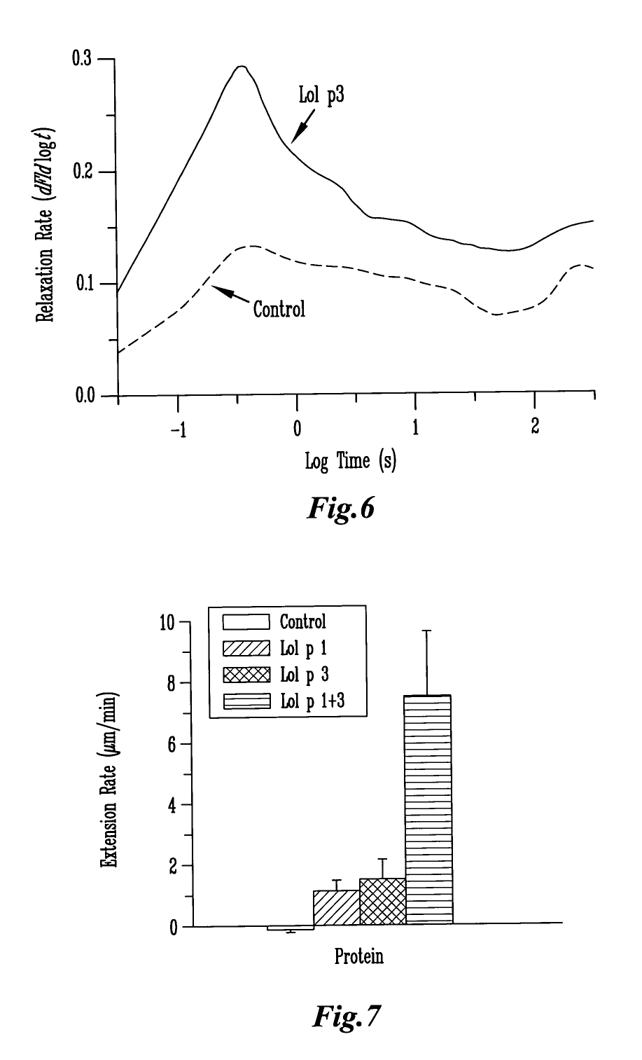

The effect of pH on wall-loosening activity of Lol p 3 is shown in FIG. 8, which shows that the Lol p 3 has an optimum pH between 4.5 and 5.5. However, outside this range there are very sharp decreases in the activity. For instance, at pH 4 and pH 6, the activity decreased by about 96% and 65%, respectively, when compared with the maximum activity at pH 5.5. This pH dependence is basically similar to that reported for both α- and β-expansins, which demonstrate that pH optima at pH 3.5-5 and 5-6, respectively, although it shows a small pH shift compared with that of α- and β-expansins.

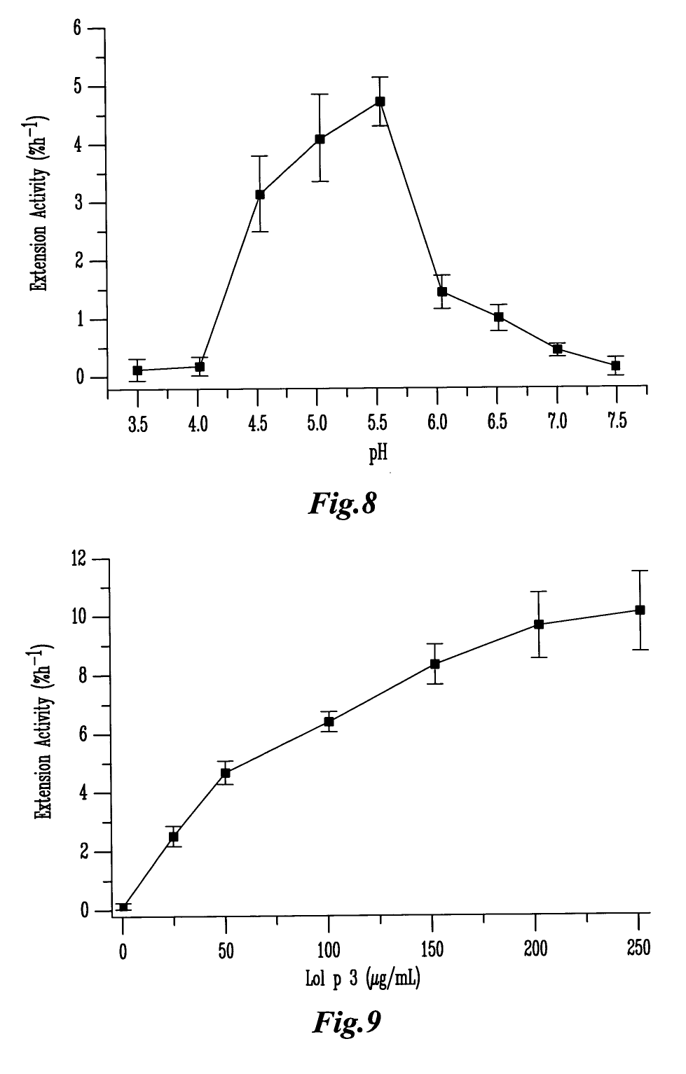

FIG. 9 shows that the wall extension activity increased as the concentration of Lol p 3 protein in the bathing buffer was increased until the concentration reached 200 μg/mL. Afterwards, it approached a saturated activity which was about two times as high as of the extension activity of native wheat coleoptile walls.

Sequence Alignment of Group 2/3 Allergens to β-Expansins

Basic BLAST searches of Genbank were performed using Lol p 3 sequences as a template. The search identified numerous related group-2/3 allergens, followed by numerous β- and α-expansins. Specifically, Applicants identified 12 group 2/3 allergens. All were from grasses, and analysis of the maize and rice EST databases indicates that they are predominantly, if not exclusively, expressed in pollen, not in vegetative tissues. It is noteworthy that the Arabidopsis genome does not contain any genes encoding group-2/3 allergens, nor are any found in current EST collections from dicots. Thus, their phylogenetic distribution indicates that group-2/3 allergens evolved only in the grass lineage, most likely by deletion of domain 1 from a β-expansin gene.

Using Clustal Method of MegAlign of DNAstar software (Madison, Wis.), the sequence identity between group 2/3 allergens and the carboxyl terminus of β-expansins was found to be up to 50% (data not shown).

Structure of Lol p 3

Recently Fedorov et al. (1997) resolved the crystal structure of a group-2 allergen (Phl p 2), which was further confirmed by solution structure (De Marino et al., 1999). Based on this structure, a predicted structure for Lol p 3 was calculated by the molecular replacement approach. On the protein surface there are a number of aromatic residues that could function in polysaccharide binding, by ring stacking, for example, as was found for other carbohydrate-binding proteins. However, the surface does not closely resemble the flat binding surface reported for cellulose-binding domain, which preferentially bind to the crystalline cellulose surface.

Group 2/3 Allergens have Wall-Loosening Activity

While Lol p 1 is a member of the β-expansin family, it was expected to have wall extension activity, but this was not expected for Lol p 3. However, the evidence, taken together, shows that the observed expansin-like wall loosening activity was not caused by contamination of Lol p 3 with β-expansin Lol p 1. First, no impurities were detected in Lol p 3 by Coomassie blue staining after SDS-PAGE (FIG. 2D). Second, there was absolutely no peak at the predicated Lol p 1 position in both MALDI-TOF and ESI-TOF mass spectra of Lol p 3 sample (data not shown). Third, the Lol p 3 preparation after HPGFC was directly subject to N-terminal amino acid sequence analysis, and there was no evidence of interference from contaminating proteins. As a result, an unambiguous sequence was readily obtained throughout 20 cycles. Fourth, and perhaps most convincingly, Western blotting analysis did not detect any Lol p 1 signal in the Lol p 3 preparation (FIG. 2D). The rabbit polyclonal antibody against Lol p 2 was cross-reactive with Lol p 1 on Western blots after SDS-PAGE separation, consistent with the earlier reports. Moreover, if the antibody used for probing Lol p 3 was replaced by the monoclonal antibody against Lol p 1, no Lol p 1 signal was revealed on the lane of Lol p 3, whereas positive control Lol p 1 gave a very strong band. Therefore, it was concluded that the wall-loosening activity observed in the Lol p 3 preparation was not due to contamination with β-expansin Lol p 1, but to Lol p 3 itself. This conclusion was strongly supported by the activity of recombinant Lol p 3. Another group-2/3 allergen from ryegrass pollen, which has a much stronger binding force to CM-column than Lol p 3, and strong wall-loosening activity was also observed. This indicates that there are multiple group-2/3 isoforms in ryegrass pollen.

Group 2/3 Allergens can Synergize with β-Expansins

α-expansins and β-expansins act on different components of the wall and differ in abundance and in their role in wall mechanics in dicots versus grass. The synergism seen in FIG. 7 suggests that Lol p 1 and Lol p 3 acts on different components of the wall. When both wall components are loosened, the action is more than additive. Alternatively, β-expansins and group 2/3 allergens promote each other's activity in some way.

Production of Transgenic Plants

Isolation of new protein often opens new possibilities of application of same. Although the potential application in paper industry is emphasized in this disclosure, numerous other directions of use can be imagined. For example expansins could be used for processing of polysaccharides for control of physical properties. Hydrogen bonding is an important determinant of many physical properties of commercial products containing polysaccharides. Expansins may be incorporated into the polysaccharide products to modify hydrogen bonding and thereby modify the physical characteristics of the products. Examples include control of the viscosity and texture of polysaccharide thickeners used in foods and chemical products, control of stiffness and texture of paper products; and control of mechanical strength (e.g., tear strength) of paper products.

In order to produce a transgenic plant, a construct that includes a heterologous gene when expressed in the plant is introduced into a plant cell. DNA constructs of the invention may be introduced into the genome of the desired plant host by a variety of conventional techniques. By way of example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector, for example. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski, et al., Embo J. 3:2717-2722 (1984). Electroporation techniques are described in From, et al., Proc. Natl. Acad. Sci. USA 82:5824 (1985). Ballistic transformation techniques are described in Klein, et al., Nature 327:70-73 (1987).

Agrobacterium tumefaciens-mediated transformation techniques, including disarming and use of binary vectors, are well described in the scientific literature. See, for example Horsch, et al., Science 233:496-498 (1984), and Fraley, et al., Proc. Nat'l. Acad. Sci. USA 80:4803 (1983).

Transformed plant cells which are derived by any of the above transformation techniques can be cultured to regenerate a whole plant which possesses the transformed genotype and thus the desired phenotype. Plant regeneration from cultured protoplasts is described in Evans, et al., Protoplasts Isolation And Culture, Handbook Of Plant Cell Culture, pp. 124-176, Macmillian Publishing Company, New York, 1983; and Binding, Regeneration Of Plants, Plant Protoplasts, pp. 21-73, CRC Press, Boca Raton, 1985. Regeneration can also be obtained from plant callus, explants, organs, or parts thereof. Such regeneration techniques are described generally in Klee, et al., Ann. Rev. of Plant Phys. 38:467-486 (1987).

The construct also includes a plant promoter that is operably linked to the heterologous gene sequence, often a promoter not normally associated with the heterologous gene. The construct is then introduced into a plant cell to produce a transformed plant cell, and the transformed plant cell is regenerated into a transgenic plant. The promoter controls expression of the introduced DNA sequence to which the promoter is operably linked and thus affects the desired characteristic conferred by the DNA sequence.

It would be advantageous to have a variety of promoters to tailor gene expression such that a gene or gene(s) is transcribed efficiently at the right time during plant growth and development, in the optimal location in the plant, and in the amount necessary to produce the desired effect. For example, constitutive expression of a gene product may be beneficial in one location of the plant but less beneficial in another part of the plant. In other cases, it may be beneficial to have a gene product produced at a certain developmental stage of the plant or in response to certain environmental or chemical stimuli.

The following are examples are not meant to limit the present invention in any fashion.

### EXAMPLE 1

### Chemical Materials

Ammonium persulfate (electrophoresis reagent), Coomassie brilliant blue R-250, and Ponceau A were purchased from Sigma-Aldrich Co. (St. Louis, Mo.) while methanol (HPLC grade) was purchased from J. T. Baker (Mallinckrodt Baker, Inc., Phillipsburg, N.J.). All other chemicals used for electrophoresis, such as acrylamide, N,N′-methylenebisacrylamide, SDS, Tris, glycine, and urea, were obtained from Research Organics, Inc. (Cleveland, Ohio). Dr. David G. Klapper (Department of Microbiology and Immunology, University of North Carolins School of Medicine, Chapel Hill, N.C.) provided mouse monoclonal antibody (anti-site D) which was raised against Lol p 1. Rabbit polyclonal antibodies, which raised against natural Lol p 2 or recombinant Phl p 2, were supplied by Dr. Alessandro Sidolli (Department of Biological and Technological Research, San Raffaele Scientific Institute, Milano, Italy) and Dr. Rudolf Valenta (Institute of General and Experimental Pathology, AKH, University of Vienna, Vienna, Austria), respectively.

### EXAMPLE 2

### Plant Materials

Ryegrass (Lolium perenne) and timothy (Phleum pretense) grass pollen were purchased from Greer Laboratories, Inc. (Lenoir, N.C.). Wheat (Triticum aestivum L., cv. Pennmore winter) seeds were grown in moist Metro-Mix 360 growing medium (Scotts-Sierra Horticultural Products Co., Marysville, Ohio) at 27-29° C. in complete darkness for 3 days. Cucumber (Cucumis sativus L. cv. Burpee Pickler) seeds were grown in wet germination paper in a dark room at 27-29° C. for 4 days. Cucumber hypocotyls were quickly excised from the seedlings under room light and directly frozen at −20° C. Wheat coleoptiles were immediately cut, gently abraded by rubbing them between two fingers coated with a slurry of well washed carborundum (320 grit; Fisher Scientific Inc., Fair Lawn, N.J.), separated from primary leaves, and then stored at −20° C. prior to use. Maize ears were collected at the beginning of August 2000 from maize (Zea mays L.) plants grown in a summer field (State College, Pa.). Silks on the ears were quickly detached, abraded, excised, and stored as described above for wheat coleoptiles.

### EXAMPLE 3

### Purification of β-expansins and Group 2/3 Allergens

Purification of β-expansins and group 2/3 allergens from ryegrass pollen was performed as previously described for maize β-expansins with some modifications. Briefly, pollen samples were extracted in 50 mM NaAc/HAc (pH 4.5) for 1 hour at 4° C. The extract was centrifuged at 15,000 g at 4° C. and was first loaded onto a SP-Sepharose Fast Flow (Amersham Pharmacia Biotech AB, Uppsala, Sweden) column equilibrated in 20 mM sodium acetate, pH 4.5. The column was washed with the same buffer and then a 0˜500 mM NaCl linear gradient with the hold at final gradient (500 mM NaCl) was applied to the column to elute the bound proteins. The fractions from SP-Sepharose column chromatography were desalted and concentrated by ultrafiltration. Active fractions were pooled and then loaded onto a silica-based CM-HPLC column (4.6×250 mm, Synchropak CM300/6.5 μm, MICRA Scientific Inc., Northbrook, Ill.). Proteins were eluted with a linear gradient of 0˜650 mM NaCl in 20 mM sodium acetate, pH 4.5, in 50 minutes. Finally, the fractions containing β-expansins and group 2/3 allergen were further purified by two coupled high-performance gel filtration columns (7.8×300 mm, PROTEIN PAK 125/10 μm and PROTEIN PAK 60/10 μm, Waters Co., Milford, Mass.) with 200 mM NaCl in 20 mM sodium acetate, at pH 4.5 as elution buffer.

### EXAMPLE 4

### Cloning of Lol p 3 cDNA

Based on the N-terminal and C-terminal amino acid sequences of Lol p 3, two degenerate 24-based sense [5′ A CN(ACGT)A AR(AG)G TN(ACGT)G AY(CT)Y(CT) TN(ACGT)A CN(ACGT)G TN(ACGT)G AR(AG)-3′ (SEQ ID NO:6)] and antisense [5′ C Y(CT)Y(CT)A R(AG)TT R(AG)TA Y(CT)TC N(ACGT)GG N(ACGT)GT R(AG)TA N(ACGT)GT-3′ (SEQ ID NO:7)] oligonucleotide were designed. The temperature gradient PCR was performed with these two degenerate oliogonucleotides as primers and with the ryegrass genomic DNA which was purified from its young leaf tissue as a template. The PCR's conditions were denaturation at 94° C. for 1 minute, annealing at 35˜45° C. for 1 minute, elongation at 72° C. for 1 minute, and a final extension at 72° C. for 10 minutes after 44 reaction cycle. Agarose electrophoresis (1.5%) revealed that only two bands exist in each PCR reaction, which annealed at 43.7 and 46.6° C., respectively. The 294-bp PCR was recovered from the gel by use of the QIAquick Gel Extraction kit (QIAGEN Inc., Valencia, Calif.), and was subcloned into pCR2.1-TOPO vector. The recombinant plasmids were transformed into TOP10 One Shot chemically competent cells with a TOPO TA Cloning Kit (Invitrogen Living Science, Carlsbad, Calif.). The plasmid was purified by use of the QIAGEN QIAprep Spin Miniprep Kit and sequenced by the chain-termination method.

### EXAMPLE 5

### Expression of Lol p 3 in Escherichia coli

The cDNA insert of Lol p 3 was digested with Nde 1 and Eco R1 and ligated into double digested pET22b(+), an expression vector that contains an isopropyl β-D-thiogalactoside (IPTG)-inducible T7 promoter and an lac operator (Novagen, Inc., Madison, Wis.). The recombinant vector was transformed into E. coli JM109 competent cells. The plasmid was then purified, sequenced and transformed into Novagen AD494 (DE3) competent cells.

The Lol p 3 expressing AD494 (DE3) cells were grown in 10 mL of Luria-Bertani medium containing 100 μg/mL of carbenicillin and 15 μg/mL of kanamycin overnight at 37° C. Cells were pelleted by centrifugation at 5 000 g for 10 minutes, resuspended back in 100 mL of culture medium and grown at 37° C. until the absorbance at 600 nm was ˜0.7. For inducing Lol p 3 expression, IPTG was added into the culture to 1.0 mM. Cells were further grown for ˜3 hours at 37° C., harvested by centrifugation, resuspended in 10 mL of cold 1% CHAPS with 5 mM DTT and ⅕ tablet of the complete protease inhibitor cocktail (Roche Molecular Biochemicals, Indianapolis, Ind.) and lysed by ultrasonication. The lystate was brought to 10 mM sodium acetate, pH 4.5, and centrifuged at 12 000 g for 20 minutes. The supernatant was chromatographed on a CM-Sepharose column and the active fraction was desalted by ultrafiltration as described for purification of natural Lol p 3. The partially purified enzyme was directly used for creep assay or stored at −20° C.

### EXAMPLE 6

### Wall Extension Assay

Expansin activity was measured with a constant load extensometer as described by Cosgrove (1989). Briefly, Abraded wheat coleoptiles and maize silks prepared as above were boiled in distilled water for 15 seconds to inactivate the endogenous α-expansins, and then secured between two clamps (with 5 mm between the clamps) under a 20-g weight of constant tension. β-expansins and group 2/3 allergen were added into the 0.15 mL-cuvette of extensometer in an appropriate buffer after the tissues were initially bathed in the same buffer for about 30 minutes. Frozen stored cucumber hypocotyls were quickly abraded with carborundum to disrupt the cuticles before complete thawing, then heat-inactivated and used as described above for wheat coleoptiles. Expansin creep activity was assayed with wheat coleoptile walls otherwise indicated, since they were easier to prepare, harder to break, had lower baseline creep rates, and proved to be a good substitute for maize silk, a natural substrate for Zea m 1.

### EXAMPLE 7

### Stress-Relaxation Measurements

Wheat coleoptiles were held between two special clamps (5 mm between the jaws) in a custom-made tensile tester as described by Cosgrove (1989). Briefly, the walls were pretreated for 10 minutes in either 50 mM sodium acetate, pH 4.5 or 0.05 mg/mL Lol p 3 and then stored on ice before stress-relaxation measurements. The tissue segments were extended at a rate of 170 mm/min until a stress of 20 g was attained and then held at a constant strain. Stress was recorded over 5 minutes by a computer with a minimum sampling rate of 2 ms, gradually increasing to 2 seconds. The relaxation spectrum was calculated as the derivative of the stress with respect to log (time).

### EXAMPLE 8

### SDS-PAGE

SDS-PAGE was performed in a mini-gel apparatus (Protean II; Bio-Rad Laboratories, Hercules, Calif.) using 12% and 15% polyacrylamide gel for Lol p 1 and Lol p 3, respectively, according to the method of Laemmli (1970). Mini-gels containing proteins were stained with Coomassie Brilliant Blue R-250 in 10% acetic acid and 30% methanol. After destaining in the same solution without the dye, gels were soaked in 4% glycerol and 30% methanol and wrapped with cellophane. Gels were scanned using an Epson flatbed scanner driven by the Photoshop software 5.5 on an IBM-compatible computer, and the molecular weight of all proteins was estimated with Kodak Digital Science ID image analysis software (Eastman Kodak Co., Rochester, N.Y.). Perfect protein marker (15-150 kD) used for SDS-PAGE was purchased from Novagen Inc. Madison, Wis. The GIBCO BRL Life Technologies, Inc. (Gaithersburg, Md.) supplied low molecular weight protein standards which consisted of alpha chain of insulin (2.3 kD), beta chain of insulin (3.4 kD), bovine trypsin inhibitor (6.2 kD), lysozyme (14.3 kD), β-lactoglobulin (18.4 kD), carbonic anhydrase (29.0 kD), and ovalbumin (43.0 kD).

### EXAMPLE 9

### Western Blotting

After SDS-PAGE, proteins were electrophoretically transferred on an EC140 Mini Blot Module (E-C Apparatus Corporation, Holbrook, N.Y.) to a Protran BA nitrocellulose membrane (Schleicher & Schuell; Keene, N.H.). Transfers were carried out in a solution of 192 mM glycine, 25 mM Tris, and 20% (v/v) methanol at 25 V for 1.5 hours. After electrotransfer, membranes were stained with Ponceau S solution for protein detection. For immunodetection of β-expansin and group 2/3 allergen proteins, the membranes were blocked with 10% horse serum in phosphate-buffered saline containing 0.05% Tween-20 and 5 mM sodium azide (PBST), incubated for 2 hours with the same solution containing mouse monoclonal antibody (anti-site D) against Lol p 1 (1:200,000 dilution) or rabbit antiserum raised against natural Lol p 2 (1:5,000 dilution) or recombinant Phl p 2 (1:2,500 dilution), washed twice with PBST and Tris-buffered saline containing 0.05% Tween-20 and 5 mM sodium azide (TBST), respectively, and then incubated for 1 hour with goat anti-mouse IgG (whole molecule)-alkaline phosphatase conjugate (dilution of 1:1000; Sigma-Aldrich Co., St. Louis, Mo.), or goat anti-rabbit IgG (heavy and light chains)-conjugated alkaline phosphatase (dilution of 1:10,000; Rockland, Gilbertsville, Pa.). The protein-containing membranes were developed with 5-bromo-4-chloro-3-indolyl phosphate/nitro blue tetrazolium (Sigma-Aldrich Co., St. Louis, Mo.). Both prestained protein ladder (15-150 kD) and low molecular weight protein standards (2.3-43.0 kD) for Western blot were purchased from GIBCO BRL Life Technologies.

### EXAMPLE 10

### Matrix-Assisted Laser Desorption/Ionization Time-of-Flight and Electrospray Ionization Mass Spectrometry

The highly purified group 2/3 allergen Lol p 3 was analyzed by the Mass Spectrometry Center of the Department of Chemistry, The Pennsylvania State University (University Park, Pa.). Matrix-assisted laser desorption/ionization time-of-flight (MALDI-TOF) mass spectra were obtained on a Voyager-DE™ MALDI-TOF (PerSpective Biosystems, Foster, Calif.), while electrospray ionization mass spectrometry (ESI-MS) was carried out on Mariner™ Electrospray-TOF workstation (PerSpective Biosystems) which was coupled with a microbore HP 1100 Series high-performance liquid chromatography (Hewlett-Packard Co., Kennett Square, Pa.). For MALDI-TOF analysis, one μL of the protein sample containing Lol p 3 was dissolved into water/methanol/acetic acid/(49:50:1 on the base of volume) at ˜2 pmol/μL, and then mixed with 1 μL of 10 μg/mL sinapinic acid in acetonitrile/H₂O/trifluoroacetic acid (70:29:0.1) as a matrix solution. One μL of this mixture was deposited on the target plate and dried to form uniform crystals. Spectra were accumulated from 76 laser shots (nitrogen laser, 337 nm). For ESI-TOF analysis, the Lol p 3 protein was automatically loaded into the HPLC, linearly eluted using water/acetonitrile/formic acid solvent system (pH 2.5) from a microbore column (Vydac C4, 1×50 mm, Keystone Scientific Co., Bellefonte, Pa.) and directly introduced into the ESI-TOF system. The system was carefully calibrated with sodium fluoride solution prior to sample analysis.

### EXAMPLE 11

### N-Terminal Amino Acid Sequence Analysis

Lol p 3 eluted from high-performance gel filtration chromatography was directly subjected to N-terminal amino acid sequence analysis at the Macromolecular Core Facility of the College of Medicine, The Pennsylvania State University (Hershey, Pa.). Approximately 1 nmol of Lol p 3 was dissolved in a minimal amount of neat trifluoroacetic acid (TFA). The protein solution was then spotted directly onto a PVDF membrane for sequencing. After TFA was removed by applying a vacuum, the N-terminal amino acids were sequenced by automated Edman degradation on an Applied Biosystems Model 477A protein microsequencer, equipped with on-line 120A high-performance liquid chromatography for analyzing the phenylthiohydantoin (PTH) amino acid derivatives.

### EXAMPLE 12

### Protein Assay

Proteins were quantified calorimetrically with the Coomassie Plus Protein Assay Reagent (Pierce, Rockford, Ill.) according to the manufacturer's instructions.

The publications and other materials used herein to illuminate the background of the invention or provide additional details respecting the practice, are herein incorporated by reference in their entirety.

It should be understood that the foregoing relates only to preferred embodiments of the present invention and that numerous modification or alterations may be made therein without departure from the spirit and scope of the invention as set forth in the appended claims.

What is claimed is:

1. An isolated nucleic acid molecule comprising a polynucleotide selected from the group consisting of:
(a) a polynucleotide or a conservatively modified variant thereof having 95% sequence identity to SEQ ID NO:1;
(b) a polynucleotide or a conservatively modified variant thereof having the sequence of SEQ ID NO:1;
(c) a polynucleotide or a conservatively modified variant thereof that encodes a polypeptide having 95% sequence identity to SEQ ID NO:2;
(d) a polynucleotide or a conservatively modified variant thereof that encodes a polypeptide that retains similar biological activity as the unmodified sequence of SEQ ID NO:2;
(e) a polynucleotide encoding a polypeptide of SEQ ID NO:2;
(f) a polynucleotide that hybridizes under high stringency conditions to the polynucleotide of SEQ ID NO:1; and
(g) a polynucleotide complementary to a polynucleotide of (a) through (f).

2. A recombinant expression cassette comprising the isolated nucleic acid molecule of claim 1.

3. A vector comprising the recombinant expression cassette of claim 2.

4. A host cell comprising the vector of claim 3.

5. The isolated polynucleotide of claim 1 wherein the polypeptide has expansin activity.

6. A group 2/3 allergen encoding a polypeptide selected from the group consisting of: SEQ ID NO:2, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18.

7. A group 2/3 allergen encoding a polypeptide comprising SEQ ID NO:2.

8. An isolated polypeptide comprising a polypeptide selected from the group consisting of:
(a) a polypeptide or a conservatively modified variant thereof having 95% sequence identity SEQ ID NO:2;
(b) a polypeptide or a conservatively modified variant thereof having the amino acid sequence of SEQ ID NO:2;
(c) a polypeptide or a conservatively modified variant that that retains similar biological activity as the unmodified sequence of SEQ ID NO:2; and
(d) a polypeptide which is encoded by the polynucleotide of SEQ ID NO: 1.

9. An antibody which selectively binds to the polypeptide of claim 8.

10. An isolated polynucleotide comprising a nucleotide sequence of SEQ ID NO: 1, and which encodes a protein having expansin activity.

11. An isolated polynucleotide having at least 95% sequence similarity to SEQ ID NO: 1 and which encodes a protein having expansin activity.

12. An isolated polynucleotide that encodes a polypeptide of SEQ ID NO:2 wherein the polypeptide has expansin activity.

13. A group 2/3 allergen isolated from grass pollen wherein the allergen possesses expansin activity.

14. A group 2/3 allergen isolated from grass pollen wherein the allergen possesses expansin activity and has an N-terminal amino acid sequence set forth in SEQ ID NO:5.

15. An isolated group 2/3 allergen having expansin activity and more than one aromatic residue on its protein surface.

16. An isolated group 2/3 allergen that has the ability to enhance the wall-loosening activity of a β-expansin in plant wall extension and stress relaxation activity.

17. The group 2/3 allergen of claim 16 wherein the enhancement is synergistic.

18. The group 2/3 allergen of claim 16 wherein said protein has wall loosening activity by itself.

19. The group 2/3 allergen of claim 18 wherein the group 2/3 allergen is Lol p 3.

20. A group 2/3 allergen that possesses expansin activity and is not affected by dithiothreitol (DDT).

21. A group 2/3 allergen having expansin activity and at least 40% sequence similarity to a carboxy terminus of a grass pollen group 1 allergen.

22. A method of modifying cells walls in the tissues of a transgenic plant, the method comprising:
introducing into a plant an expression cassette compromising a promoter active in cells of plants operably linked to a group 2/3 allergen polynucleotide which specifically hybridizes to SEQ ID NO:1 under stringent conditions.

23. A method of weakening the mechanical strength of cellulose fibers, the method comprising:
contacting a quantity of cellulose with a composition having a polypeptide comprising an amino acid sequence of SEQ. ID. NO:2.

24. A method of modifying plant cell walls, the method comprising:
introducing into a plant a polynucleotide sequence that encodes a polypeptide sequence comprising SEQ ID NO:2, the method comprising:
cultivating the plant under conditions suitable for plant growth and production of the polypeptide;
harvesting the plant; and
recovering the polypeptide.

25. A method for producing a polypeptide having expansin activity comprising:
(a) cultivating the host cell of claim 4, under conditions suitable for production of the polypeptide; and
(b) recovering the polypeptide.

26. A transgenic plant cell comprising a nucleic acid comprising the sequence of SEQ ID NO:1.

27. A transgenic plant with a genome comprising a nucleic acid comprising the sequence of SEQ ID NO:1 that possess expansin activity.

28. Seeds of the plant of claim 27 which carry the DNA construction in their genome.

29. A transgenic plant comprising an expression cassette operably linked to a group 2/3 allergen polynucleotide which specifically hybridizes to SEQ ID NO:1 under stringent conditions.


SEQUENCE LISTING (18 sequences)

SEQ ID NO: 1 - DNA, 291 bp, Lolium perenne
acaaaagtcgatttaactgtggagaagggttctgacgcgaagacgctggtgctgaacatcaagtacacgaggccaggggacaccctggcggaggtggagctccggcagcacggctcggaggagtgggaacccatgacgaagaagggcaacctgtgggaggtgaagagcgccaagccgctcaccggcccaatgaacttccgcttcctctccaagggcggcatgaagaacgtcttcgacgaggtcatccccaccgccttcacggtcggcaaaacctacaccccagaatacaat

SEQ ID NO: 2 - protein, 97 aa, Lolium perenne
TKVDLTVEKGSDAKTLVLNIKYTRPGDTLAEVELRQHGSEEWEPMTKKGNLWEVKSAKPLTGPMNFRFLSKGGMKNVFDEVIPTAFTVGKTYTPEYN

SEQ ID NO: 3 - DNA, 291 bp, Lolium perenne
  misc_feature (133)..(133): Conservatively modified variant; C=substituted nucletide
acaaaagtcgatttaactgtggagaagggttctgacgcgaagacgctggtgctgaacatcaagtacacgaggccaggggacaccctggcggaggtggagctccggcagcacggctcggaggagtgggaacccctgacgaagaagggcaacctgtgggaggtgaagagcgccaagccgctcaccggcccaatgaacttccgcttcctctccaagggcggcatgaagaacgtcttcgacgaggtcatccccaccgccttcacggtcggcaaaacctacaccccagaatacaat

SEQ ID NO: 4 - protein, 97 aa, Lolium perenne
  MISC_FEATURE (83)..(83): Conservatively modified variant; A=Substituted amino acid at position 83 (proline, cca to alanine, ccc).
TKVDLTVEKGSDAKTLVLNIKYTRPGDTLAEVELRQHGSEEWEPMTKKGNLWEVKSAKPLTGPMNFRFLSKGGMKNVFDEVIATAFTVGKTYTPEYN

SEQ ID NO: 5 - protein, 20 aa, Lolium perenne
TKVDLTVEKGSDAKTLVLNI

SEQ ID NO: 6 - DNA, 24 bp, Artificial Sequence
  Degenerate oliogonucleotide design based on the N-terminal and C-terminal amino acid sequences of Lol p 3.
acnaargtngayytnacngtngar

SEQ ID NO: 7 - DNA, 25 bp, Artificial Sequence
  Degenerate oliogonucleotide design based on the N-terminal and C-terminal amino acid sequences of Lol p 3.
cyyarttrtaytcnggngtrtangt

SEQ ID NO: 8 - protein, 93 aa, Zea mays
TFQVGKGSKPGHLVLTPNIATISDVEIKEHGGDDFSFTLKEGPAGTWTLDTKAPLKYPLCIRFATKSGGYRIADDVIPADFKAGTTYKTTLSI

SEQ ID NO: 9 - protein, 96 aa, Dactylis glomerata
VKVTFKVEKGSDPKKLVLDIKYTRPGDTLAEVELRQHGSEEWEPLTKKGNLWEVKSSKPLTGPFNFRFMSKGGMRNVFDEVIPTAFKIGTTYTPEE

SEQ ID NO: 10 - protein, 90 aa, Oryza sativa
MEVAKGSSAKSLELVTNVAISKVEVKEKGGKDWVALKESSSNTWTLKSESPLKGPFSVRFLVKNSGYRVVDDIIPESFTAGSEYKSGIQL

SEQ ID NO: 11 - protein, 96 aa, Lolium perenne
AAPVEFTVEKGSDEKNLALSIKYNKEGDSMAEVELKEHGSNEWLALKKNGDGVWEIKSDKPLKGPFNFRFVSEKGMRNVFDDVVPADFKVGTTYKP

SEQ ID NO: 12 - protein, 96 aa, Sorghum
GTTLTIEVGKDSTSTKLSLITNVAISEVSVKPKGATDFTDDLKESEPKTFTLDSKEPIEGPIAFRFLAKGGGYRVVDNAIPADFKAGSVYKTTEQV

SEQ ID NO: 13 - protein, 100 aa, Hordeum vulgare
AATKVKFTVQKGSDAKKLVLKIDYTRAGDTLSEMELRQHGSEEWEPFTKKGDVWELSSSKPLVGPFNFRFLSKGGMKNVFDEVFSTDFKIGKTYEPVYDA

SEQ ID NO: 14 - protein, 98 aa, Triticum aestivum
APPPVSITVEKGSDAKHLVLQIKYDKVGDSMKEVELEQNEDWLPLKKGYSGAWEIKSDTPLKGPFSFRYETQKGQRNVFDDIVPTDFKCGTTYKPEAY

SEQ ID NO: 15 - protein, 96 aa, Phleum pratense
VPKVTFTVEKGSNEKHLAVLVKYEGDTMAEVELREHGSDEWVAMTKGEGGVWTFDSEEPLQGPFNFRFLTEKGMKNVFDDVVPEKYTIGATYAPEE

SEQ ID NO: 16 - protein, 98 aa, Hordeum vulgare
AVPPVSFTVEKGSEEKKLALQIKYDKEGDSMKEVEVKQGEEWLPLNKCANGVWEIKVDEPLKGPYSIRYETDKGQRNVFDDVVPAEYKIGTTYKPAEP

SEQ ID NO: 17 - protein, 97 aa, Triticum aestivum
  MISC_FEATURE (17)..(17): X is unknown.
AVRVKLTVEKGSDKKKLALKIDYTRPXDSLSEVELRQHGSKEWQPVTKNGDVWEVSCSKPLVGPFNFRFLSKNGMKNVFDEVFSTDFKIGKTYQPEY

SEQ ID NO: 18 - protein, 96 aa, Triticum aestivum
VKVKLTVQKGSDKKKLALKIDYTRPNDSLSEVELRQYGSEEWQPLTKKGDVWEVSCSKPLVGPFNFRFLSKNGMKKVFDEVFSTDFKIGKTYEPEY